(19) United States
(12) Patent Application Publication
(10) Pub. No.: US 2021/0132057 A1
(43) Pub. Date: May 6, 2021

(54) New test kit containing pancreatic elastase-1-specific antibodies and a sample preparation device

(71) Applicant: SCHEBO BIOTECH AG, Giessen (DE)

(72) Inventors: Hans SCHEEFERS, Wettenberg (DE); Ursula Scheefers-Borchel, Wettenberg (DE)

(21) Appl. No.: 17/016,700

(22) Filed: Sep. 10, 2020

(30) Foreign Application Priority Data
Oct. 12, 2018 (DE) 102018008075.8

(51) Int. Cl. G01N 33/543; B01L 3/00; G01N 1/04

(52) U.S. Cl. CPC G01N 33/54386; B01L 3/523; B01L 2200/10; B01L 2300/0627; G01N 1/04

(57) ABSTRACT

The present invention relates to a test kit comprising a pancreatic elastase 1-specific antibody with a sample collection device and an extraction buffer, which surprisingly has a higher accuracy of determination of the elastase 1 in the feces.

### BACKGROUND OF THE INVENTION

The present invention relates to a test kit with pancreatic elastase 1-specific antibodies and a novel sample preparation device for dosage and extraction, which surprisingly permits a more precise detection of pancreatic elastase 1 in the feces.

### PRIOR ART

It is known to detect inflammatory diseases of the pancreas by qualitative and quantitative determination of the pancreatic elastase 1 (in the following also: E1). The EP 0547059 B1 (SCHEBO TECH MEDIZINISCH-BIOLOGISCHE FORSCHUNGSGESELLSCHAFT M.B.H) describes, inter alia, an immunological test kit, which comprises antibodies against the pancreatic elastase 1. According to this document, body liquids can be examined for E1 by means of different test kits comprising anti-pancreatic elastase 1-antibodies. This is achieved, for instance, for blood, plasma, serum, but also feces. Such tests are used since many years for the examination of feces and serum samples for E1.

Surprisingly, it has been found out that a test kit according to claim 1 permits a clearly more precise determination of E1 in the feces than test kits of the prior art.

The test kit comprises, first of all, a sample preparation device, by means of which, e.g., feces samples to be enzymatically or immunochemically analyzed, can be prepared for the analysis.

The sample preparation device according to the invention permits an exact dosage and extraction of feces samples in a liquid reagent or/and a liquid solvent and is sealed, so that it can also be subjected to an underpressure during a potential air transport.

The sample preparation device according to the invention consists of a closeable vessel for receiving a liquid reagent or/and liquid solvent and a sample collection rod, which has in the region of one of its ends on its peripheral surface at least one scoop-type recess, and which is insertable with this end into the vessel via an insertion opening of the vessel provided with a stripping shoulder. Such a sample preparation device is, in principle, already described in the German utility model DE 2032160U1. It has been found, however, that the shape of the scoop-type recess strongly affects the accuracy of the measurement. The majority of the feces has a very heterogeneous composition and a high proportion of fibers. The E1 to be quantified is found in the slimy (that is, in the non-fibrous) portion of the feces only. In the fibrous portion of the feces, there is practically no E1.

### SUMMARY OF THE INVENTION

The technical object to be solved is, therefore, to separate the slimy portion of the feces from the fibrous portion. This object is technically solved by the use of a sample preparation device, which, according to the following description, comprises specially developed scoop-type recesses. By the use thereof, practically only the slime portion of the feces is supplied to the extraction. In the majority of the extract, there is practically no fibrous portion, which would falsify the result of the calculation. While not wishing to be bound by any particular theory, the inventors assume that by the design of the scoop-type recesses described in the following, the fibrous portion of the removed feces sample is reduced to a minimum. Since the E1 to be determined by means of the invention is mainly comprised in the slime portion of the feces and practically almost never in the fibrous portion, a different fibrous portion strongly influences the accuracy. By the design of the scoop-type recess(es) described in the following, the fibrous portion is reduced to a minimum, so that the measurement can be performed as precisely as possible. Further, it is already known from the literature that very aqueous feces, due to the dilution effect, may lead to reduced elastase 1 concentrations in the feces (low E1 concentrations=pathologic result). By the sample preparation system, only suitable feces are collected, this leads to an optimum feces—buffer concentration, the optimized concentration leads to a higher precision.

### DETAILED DESCRIPTION OF THE INVENTION

According to the invention, the scoop-type recess is formed by one or several annular or ring segment-shaped recesses in a cylindrical sample collection rod. The sample collection rod typically has a diameter d of 1-2 mm. An the end of the sample rod, which is introduced into the feces, the cylinder is provided with a tip (not shown in the figures). At the opposite end, there are the handle and the closure elements (not shown in the figures).

In principle, the sample collection rod may comprise an annular recess (FIG. 1). It has been found that the angle α between the lateral surface of the sample collection rod and the recess should be approximately rectangular. Further, the angle β between the base surface of the recess and the core cylinder should also be approximately rectangular. The depth t of the recess should not be deeper than 0.1 mm, the height h of the recess should not be higher than 0.5 mm. The total volume of such a recess should not exceed 20 μl.

Further, it has been found that a completely annular recess of the scoop-type recess is not optimum, since the core cylinder resulting from the recess may easily be bent or even broken by the forces generated during the reintroduction of the sample collection rod into the vessel, there is created so to speak a predetermined breaking point. By a not completely annular recess, but by a ring segment-shaped recess (FIG. 2), the sample collection rod gains mechanical stability, while at the same time the total volume of the recess decreases. The recessed ring segment may span, for instance, 270° (FIG. 2), 180° (FIG. 4) or 90° (FIG. 3) of the cylinder cross-section. An optimum compromise between mechanical stability of the sample collection rod and the volume of the recess is an angle of 180° (FIG. 4).

The volume of the recess corresponds, of course, to the volume of the sample, if only one single recess is provided. Should the desired sample volume be greater than 20 μl, expediently several recesses are provided, e.g., 2 to 6 recesses (see, e.g., FIG. 5 with 3 recesses). For the purpose according to the invention, it has been found that a sample volume of 50 μl is optimum, which is formed by four recesses of 12.5 μl each.

The closeable sample preparation device comprises 1-2 ml of a buffer solution. The buffer solution comprises a phosphate-buffered sodium chloride solution with a pH value of 6.9-8, Tween 20 and sodium azide <0.05%. In order to obtain a complete extraction, it is important to admix to the phosphate-buffered extraction buffer the detergent CHAPS (3-[(3-cholamidopropyl)dimethylammonio]-1-propanesulfonate, 10 mM (Sigma).

Further, by the sample preparation device, an accelerated and more hygienic working process is possible for the user; a time-consuming step in the working process of the test kit can be omitted, since the feces need not be weighed, but are dosed based on the geometry of the cavities according to the invention. Another advantage of this use is that, different from prior art, now even smaller feces amounts are required for the measurement.

The test kit according to the invention further comprises a device for the in vitro measurement of E1 in the feces, wherein the device comprises at least one solid phase to which is bound at least one antibody, selected from the group of the elastase 1-specific monoclonal antibodies capable of binding, “Clone 1”.

A particular subject matter of the invention is the use of the E1-specific antibody according to the invention for the qualitative and/or quantitative determination of E1. It is possible, by using the antibody, to specifically detect the elastase 1 in body liquids and in the feces.

The invention also relates, thus, to a test kit comprising the antibodies according to the invention, and in particular to immunological test kits for the diagnosis and exclusion of an exocrine pancreatic insufficiency in the feces.

In experiments for the detection of E1 in the feces, indirect, competitive, and sandwich-ELISAs are employed. It has been found out, however, that a sandwich-ELISA is most suitable for quick diagnostics with large sample sizes. For this purpose, at least two different monoclonal antibodies are required, which are directed against different epitopes of the enzyme. By means of such tests, enzyme displacements in the feces, in particular the occurrence of these displacements with changes in the pancreas status, can quantitatively be detected.

Methods of determination based on the principle of immunoassays are widely used. The advantage of this method of determination is its accuracy and rapidity (large security and sample throughput) and the possibility to detect very small amounts of substances (in the nanogram range). For carrying-out the determination, different variants of the method are possible, with homogeneous as well as with heterogeneous phases. In the embodiment with heterogeneous phase, one of the receptors is bound to a carrier. In a sandwich-method, for instance, a first antibody is bound as a receptor, or as a so-called catcher to the carrier, the test solution is added, with the antigen to be determined in the test solution being captured by the receptors from the test solution and being bound. Then, a second, marked antibody is added that specifically reacts with the antigen or the antigen-receptor complex. By means of the marking of the second antibody, therefore, the amount of antigen can then be determined with the aid of a calibration solution (isolated, purified human elastase 1).

In another preferred embodiment according to the invention, a first antibody is bound as a receptor to a carrier matrix with a membrane, tissue or fleece structure such that it will not, as usual, represent the bottom of the recess of an Elisa immunoplate, but is bound in the matrix. Preferred matrices are microporous flat membranes or hollow fiber membranes, which, in a particular embodiment, are provided with ion exchanger groups. For this purpose, preferably such microporous flat membranes are used, as they are marketed, e.g., by Pall Corp., New Jersey, USA. The hollow fiber membranes to be used according to the invention are also commercially available and are marketed, e.g., by Sepracor Inc., Ma., USA. By means of such carrier materials, it is possible to provide particularly fast and uncomplicated detection methods.

With this general principle, there are many possibilities of variation. For instance, a determination with three receptors may be selected, wherein one of the three receptors is present in a heterogeneous phase, and the other two receptors are soluble. One of the two soluble receptors is marked, whereas the other one is non-marked. The soluble receptor is then directed against the non-marked receptor.

The use of the E1-specific antibodies according to the invention for the selective quantitative determination of E1 using the principle of the immunoassay is performed by incubation with at least two different receptors. Both receptors, e.g., monoclonal antibodies, must be specific for E1, which has to be bound via respectively different epitopes (binding sites).

One of the two receptors is bound to a solid phase. The binding to the solid phase occurs in a usual way and is known to the person skilled in the art. Further, at least one additional receptor is used that exists in a soluble form.

This additional receptor carries a marking. When several additional receptors R2 are used, only one of these carries a marking. The marking of the receptor is performed in a per se usual way and is known to the person skilled in the art.

In the test kit according to the invention, the marking is made in a per se known way, in particular by a radioactive marking, by binding biotin (biotin/avidin), by an enzyme causing a measurable reaction, or by a chemiluminescent or fluorescent compound. Particularly preferred is the marking with an enzyme, in particular with peroxidase or phosphatase. The marking with an enzyme also permits, in a particular embodiment, to use these antibodies in a second enzyme amplification system (Stanley, C. J., Paris, F., Plumb, A., Webb, A. Johannsson, A. American Biotechnology Laboratory: May/June; 1985; Self, C. H., J. Immunol. Meth.; 1985).

In a particularly preferred embodiment of the method, now either a receptor capable of non-specifically binding with E1 or preferably a receptor capable of specifically binding with E1 is bound to a solid phase. This receptor bound to the solid phase is then incubated with the solution that contains the E1 to be determined, and an antibody that is capable of specifically binding with E1, is present in a soluble form and carries a marking.

When the receptor bound to the solid phase is capable of nonspecifically binding with E1, then not only E1 accumulates at the solid phase, but also other antigens. The second antibody that is capable of specifically binding with E1, accumulates, however, only at E1, so that only E1 molecules specifically carry a marked antibody, while the other antigens are not marked. In this way, it is possible, after separation of the solid from the liquid phase, to determine the content of E1 through the measurement of the marking.

When a first receptor specifically capable of binding to E1 is attached to the solid phase, then only E1 is specifically bound to the solid phase. During the incubation with the soluble, E1-specific second receptor or antibody, the latter also reacts exclusively with E1. Since, thus, practically no binding of other antigens to the solid phase takes place, this method is highly specific and permits, therefore, very precise determinations. To the solid phase, E1 is selectively bound, other antigens remain in the solution. Further, the soluble, marked antibody capable of binding with E1 accumulates at E1. After separation of the solid phase from the liquid phase, again the content of E1 can very precisely be determined through the marking. In a particularly preferred embodiment, for further increase of the selectivity, a third antibody is added that is directed against the second antibody and that carries the marking.

Further variants of the method known to the person skilled in the art with three receptors are also possible using the antibodies capable of specifically binding with E1, which does not need any further explanations here.

Among the antibodies used for carrying-out the method according to the invention, preferably at least one is a monoclonal antibody. In a preferred embodiment, only monoclonal antibodies are used as receptors.

The antibody capable of specifically binding with E1 can either be bound to the solid phase or used as a soluble, marked or non-marked receptor. Preferably, this receptor is a monoclonal antibody. Particularly preferably, all used receptors are monoclonal antibodies.

The method according to the invention and the test kit are particularly suited for automated analysis systems, in particular for such systems that are based on a biosensor and make use of the chip technology.

### EXAMPLES

The invention is explained in more detail with reference to following examples:

### a) Production of Monoclonal Antibodies by Means of Purified E1:

Purified human E1, the extraction of which from human pancreas has been described (Sziegoleit A., Purification and Characterization of a cholesterol binding protein from human pancreas. Biochem. J. 207:573-582; 1982), is dissolved in PBS and mixed in equal portions with Freund's adjuvant. 100 μg each of this mixture are injected i.p. and s.c. into 6 to 8 weeks old Balb/c mice. These injections are repeated twice after 3 to 4 weeks. Mice immunized with purified E1 according to the above scheme obtain 3 days before removal of the spleen an i.v. injection of 100 μg of purified E1, which is dissolved in PBS.

Approx. 100 mill. cells of the spleen of an immunized mouse are fused with 50 mill. myeloma cells (x 63-Sp8-653, a cell line that does not synthesize immunoglobulin; available from The Salk Institute, Cell Distribution Center, San Diego, Calif. 92112, USA) in presence of polyethylene glycol (MG 3400). Fused cells are sown on 8 plates each comprising 24 recesses. Each of these recesses comprises 50 mill. spleen cells of non-immunized syngeneic mice in culture medium, which comprises hypoxanthine, aminopterin, and thymidine.

The supernatants of these fused cells (hybridoma) comprising antibodies are tested 10 to 14 days later by means of ELISA, western blot, frozen section, paraffin section for their specificity against highly purified, human E1.

In order to obtain monoclonal antibodies that are only directed against E1, hybridoma cells, the supernatants of which do not contain antibodies directed against other antigens, are cloned twice. Only hybridoma cells are cloned, without AB-secretion, which did not exhibit any cross-reaction with pancreatic elastase of pig and cattle.

Thereby, it can be assured that the intake of substitution preparations does not falsify the test result.

### Example 1

Determination of E1 in the Feces with Monoclonal E1-Specific Antibodies.

E1 in the feces is determined with an ELISA. For this purpose, obtained monoclonal, E1-specific antibodies are dissolved in PBS, pH 7.2 and immobilized at polystyrene as a carrier. After a washing step, extracted feces, which comprise E1, are added. The extract is diluted in a buffer that comprises PBS, 5 mmol EDTA and 0.2% Tween 20.

After a washing step in PBS, 0.2% Tween 20, the E1 bound to the antibodies is incubated with a monoclonal antibody that also binds E1 and is coupled to the phosphatase that is dissolved in PBS that comprises 0.2% Tween 20 and has a pH of 7.2, for one hour at room temperature. After another washing step by addition of p-nitrophenylphosphate disodium hexahydrate, a change of the optical density in the reaction vessels is measured, in which the monoclonal antibodies have reacted with E1.

### Example 2

Determination of E1 in the Feces with Polyclonal E1-Specific Antibodies.

E1 in the feces is determined with an ELISA. For this purpose, obtained monoclonal, E1-specific antibodies, are dissolved in PBS, pH 7.2 and immobilized at polystyrene as a carrier. After a washing step, extracted feces, which comprise E1, are added. The extract is diluted in a buffer that comprises PBS, 5 mmol EDTA and 0.2% Tween 20.

After a washing step in PBS, 0.2% Tween 20, the E1 bound to the antibodies is incubated with a monoclonal antibody that also binds E1 and is coupled to the phosphatase that is dissolved in PBS that comprises 0.2% Tween 20 and has a pH of 7.2, for one hour at room temperature. After another washing step by addition of p-nitrophenylphosphate disodium hexahydrate, a change of the optical density in the reaction vessels is measured, in which the monoclonal antibodies have reacted with E1.

### Example 3

Determination of E1 in Serum with Two Different E1-Specific Antibodies.

A monoclonal antibody that is directed against E1, is attached, same as described in Example 1, to a carrier. After a washing step, serum that comprises E1 is incubated with the monoclonal antibody under the same conditions as in Example 1. After another washing step, the binding of E1 is detected by a second E1-specific monoclonal antibody according to the invention. This second E1-specific antibody carries covalently bound peroxidase. After a washing step and after addition of ABTS as a substrate for peroxidase, a change of the optical density is measured.

### Example 4

The procedure is as described in Example 2, however, instead of an enzyme, biotin is coupled to the second E1-specific antibody. Before substrate addition, peroxidase-conjugated avidin or peroxidase-conjugated streptavidin is added. The thus selected determination of E1 permits a very specific determination of E1 only. Other antigens that are contained in the solution, will not affect the determination of the E1 according to the invention.

### Example 5

### Manufacture of the Sample Preparation Device for Dosage and Extraction.

The manufacture of the sample preparation device for dosage and extraction is achieved as shown in FIG. 6.

### Example 6

### Manufacture of the ELISA System

The manufacture of the ELISA system is achieved as shown in FIG. 7.

### Example 7

### Performing the Tests

The test is performed as shown in FIG. 8. The essential steps are:

Prepare the sample/washing buffer

Dose and extract feces with the sample preparation device

Dilute feces extract in sample/washing buffer

Pipet 50 μl each of blank, standards, positive control and samples as double determination in ELISA plate—incubate for 30 minutes at room temperature—wash

50 μl each of anti-E1-bio-POD-streptavidin complex (ready to use)—incubate for 15 minutes at room temperature in the dark—wash

100 μl substrate solution (ready to use)—incubate for 15 minutes at room temperature in the dark

Add 100 μl of stop solution (ready to use)

### Evaluation of the Tests

The OD measurement is made at 405 nm at the time of 5 to 30 min after addition of the stop solution. Prior to the measurement, the ELISA plate must be thoroughly shaken. When the measurement is performed against a reference wavelength, this should be 492 nm.

### Example 8

a) The elastase 1 test kit was tested with human feces samples with the specially developed sample preparation device in comparison with the elastase 1 test kit with the conventionally contained extraction buffer. As a predetermined value, the value measured with the ELISA and conventional extraction buffer was taken. All literature about the ScheBo elastase 1 test kit describes this as a non-invasive gold standard of the pancreas functional diagnostics, therefore, in our tests, too, the ScheBo E1 ELISA was taken as a gold standard. Now, the feces were punctured 10× with the extraction system, the elastase 1 value was determined and therefrom the mean value was calculated. This mean value was then compared to the predetermined value. The % difference of the samples from the predetermined value varied between −14 and 4%.

There are also other known suppliers, who offer such sample preparation devices; the same tests were made with the sample preparation device of another leading manufacturer. For this purpose, again the feces were punctured 10× with the sample preparation device. The measurement results are shown in FIG. 10. The % VarK (also % CV) of the measured elastase 1 values varied between 11.01 and 32.34%, wherefrom followed a % difference of the samples to the predetermined value between −23 and 44%. It is thus clearly recognizable that the precision of the sample preparation device contributes a very decisive part to the test process. However, the calculation of the samples that were processed with the sample preparation device of a competitor, yielded a lower value. This is particularly critical for samples around the cut-off of 200 μg/g feces, since they are incorrectly pathologically graded/classified.

b) The enclosed drawings (FIGS. 9, 10) illustrate the very high precision of the sample preparation device according to the invention. The measurements of the intra- as well as of the inter-coefficient of variation (VarK or CV)) yield a very high degree of accuracy. For determination of the intra- and inter-VarKs, with two sample preparation systems, for two different feces samples in each feces sample, 30 times subsequently the amount of feces taken-up with the system was measured. The sample preparation device exhibits here excellent 10% VarK values. The intra-% VarK value is for one tube 2.6610% VarK and for the others nearly identical 2.6440% VarK. The inter-VarK resulting therefrom between two tubes with different feces samples is just 0.3205% VarK, which is also an excellent result. The agreement of both tubes is thus at 99.6795%.

### DESCRIPTION OF THE FIGURES

FIG. 1 shows a sample collection rod according to the invention with an annular recess.

FIG. 2 shows a sample collection rod according to the invention with a ring segment-shaped recess, spanning 270° of the cylinder cross-section.

FIG. 3 shows a sample collection rod according to the invention with a ring segment-shaped recess, spanning 90° of the cylinder cross-section.

FIG. 4 shows a sample collection rod according to the invention with a ring segment-shaped recess, spanning 180° of the cylinder cross-section.

Therein are:

d: outer diameter;

d2: diameter of the core cylinder formed by producing the scoop-type recess;

t depth of the recess;

h height of the recess;

α angle between the lateral surface of the sample collection rod and the recess;

β angle between the base surface of the recess and the core cylinder;

FIG. 5 shows a sample collection rod with three ring segment-shaped recesses according to the invention, each spanning 180° of the cylinder cross-section;

FIG. 6 shows the plan of operations for the manufacture of the sample preparation device for dosage and extraction.

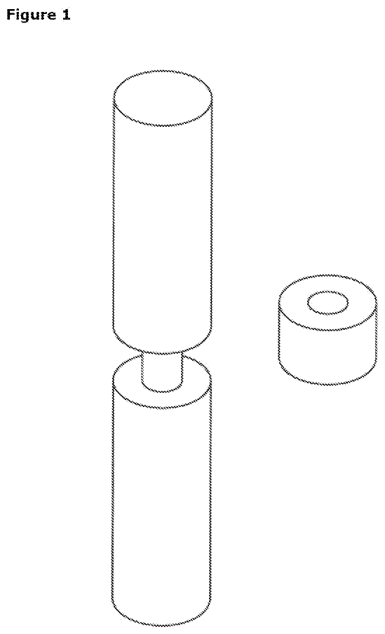

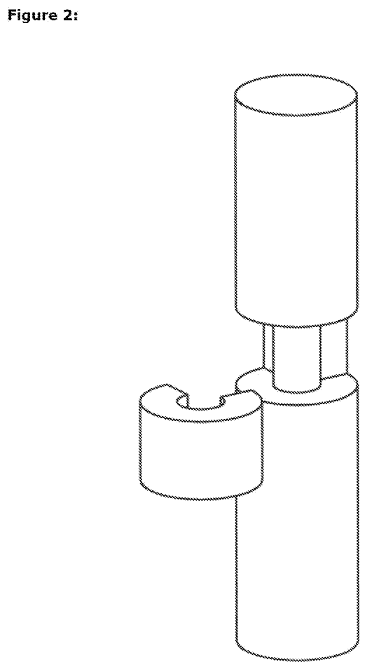

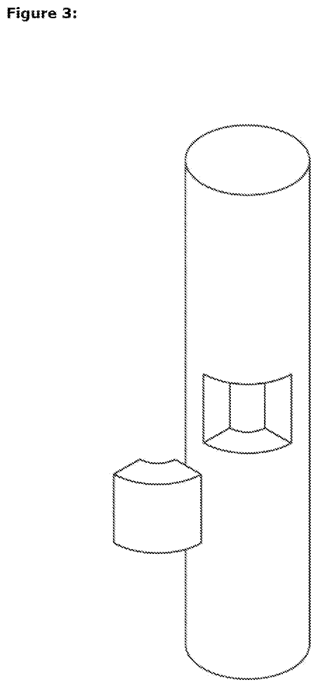

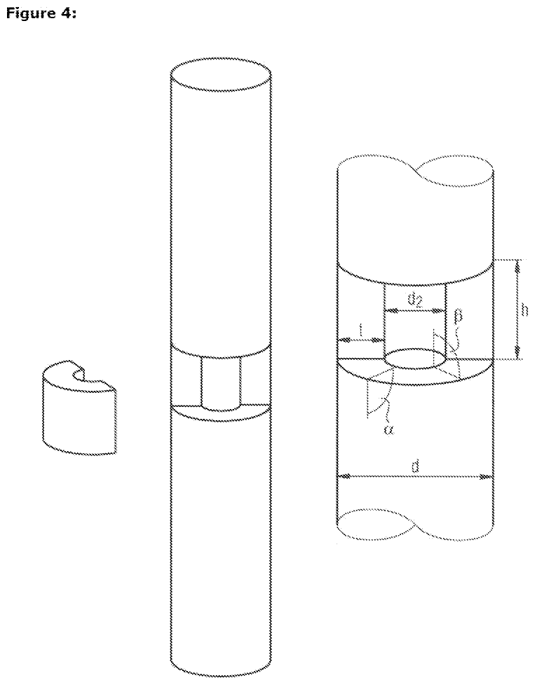

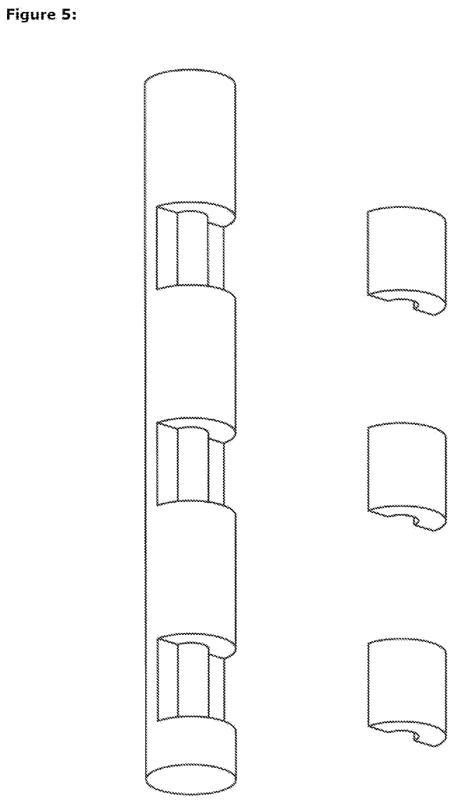

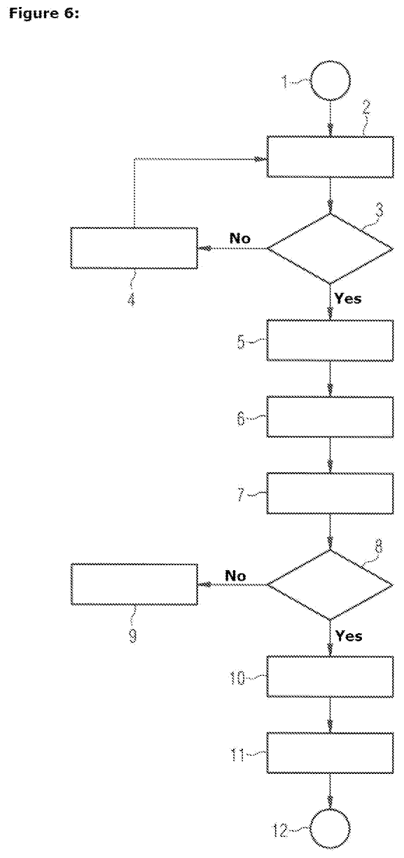

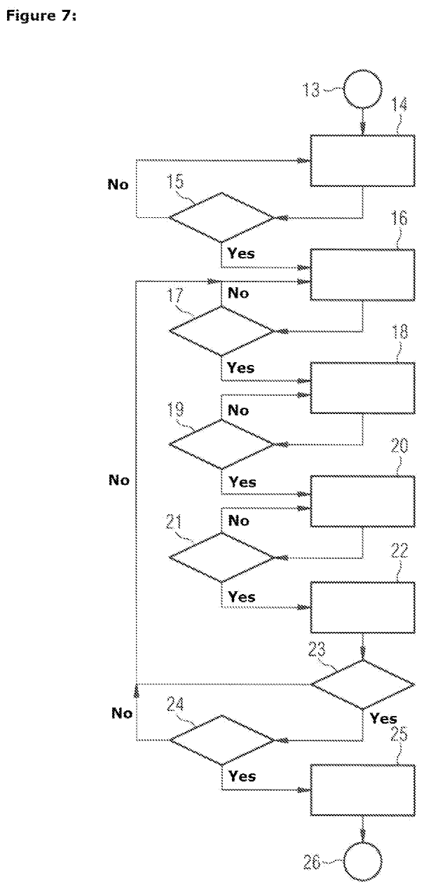

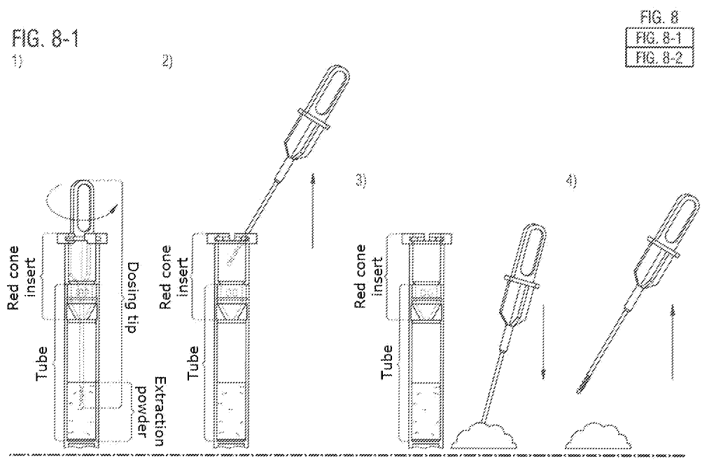

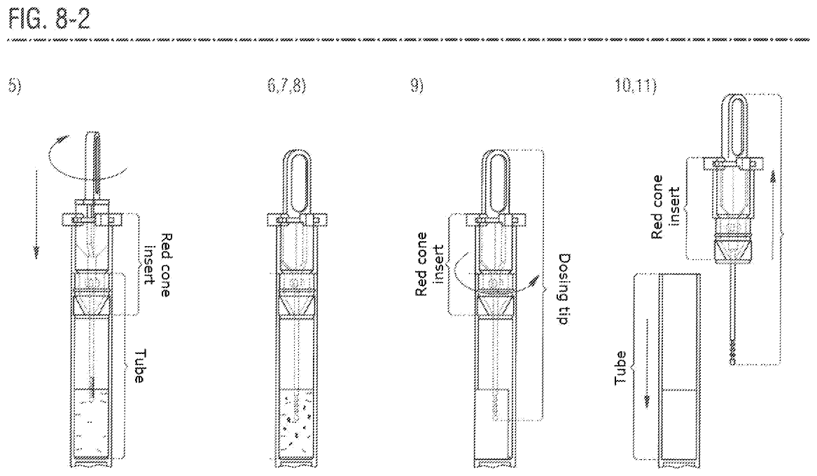

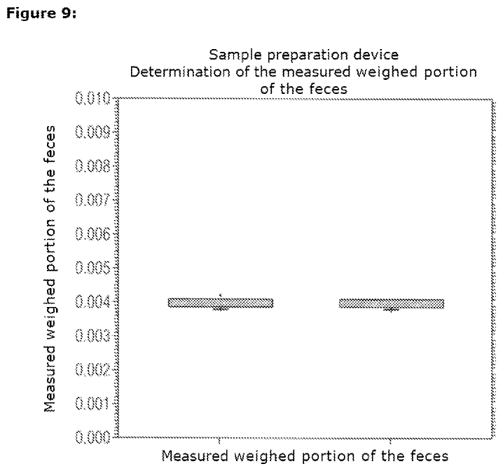

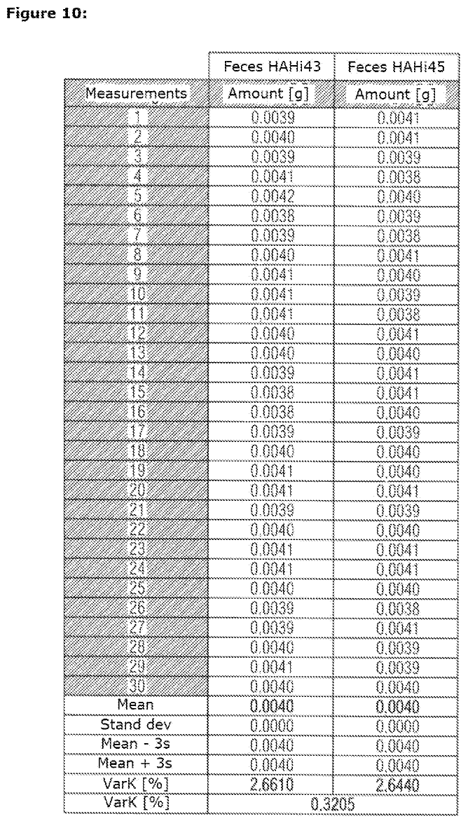

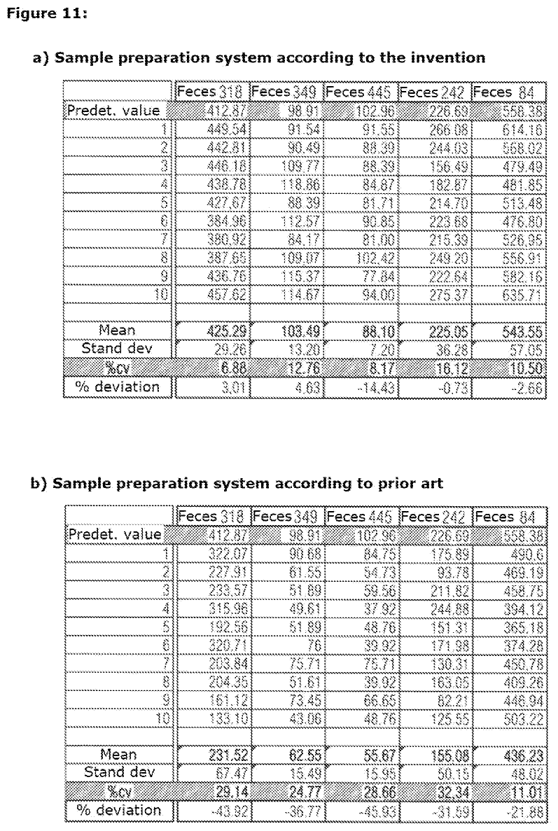

Therein are:
1: start;
2: delivery: sampling rod, cone as stripping shoulder and tube;
3: quality control o.k.?; (inspection of dosing tip, of the cone and of the tube for production faults, dust, fluffs, dirt or the like. Approval of the material, if suitable);
4: return to manufacturer;
5: sampling rod is introduced into cone;
6: tube is filled with extraction buffer;
7: sampling rod with cone is introduced into tube with extraction buffer;
8: quality control o.k.? (test for leakages);
9: rejection;
10: packing;
11: storing;
12: end.

FIG. 7 shows the plan of operations for the manufacture of the ELISA system; Therein are:
13: start;
14: coating the plates with monoclonal anti-elastase 1 antibody;
15: quality control o.k.? Test of randomly selected plates with control feces samples;
16: production of the test reagents;
17: quality control of the test reagents;
18: in-house production of the packing labels;
19: inspection and production of the other materials (e.g., foil, bottles, cartons, caps) o.k.?;
20: filling the tested reagents into bottles in the manufacturer's lab;
21: inspection of the filled and labeled bottles o.k.?;
22: packing of the kits and storing at 4° C.;
23: quality control of the stored kits (every quarter of a year), o.k.?;
24: stability control at 37° C., o.k.?;
25: storing and shipping of approved kits;
26: end.
   FIGS. 8.1 and 8.2 show the application of the sample collection device. Therein are:
1) turn sample collection device toward left (unlock);
2) pull sample collection device out;
3) puncture the feces sample with the sample collection device approx. 1 cm deep (all scoop-type recesses must be filled with the feces);
4) pull sample collection device out of the feces;
5) fit the sample collection device by the cone insert (stripping shoulder) into the tube and turn the sample collection device for locking toward right;
6) mix thoroughly (test tube shaker);
7) extract for ten minutes;
8) then mix. Attention! No feces anymore at the sample collection device!;
9) turn cone insert for opening the tube toward left and press the cone insert slightly upward until “crack”;
10) pull cone insert together with the sample collection device out of the tube;
11) after deposition of the particles, the feces sample extract from the tube is diluted 1:90: 10 μl feces sample extract+900 μl sample/washing buffer 1×;

The cone insert of the tube serving as a stripping shoulder can be colored for better detectability (here: red). The color only serves for the easier handling by the user and may also be designed differently. Corresponding considerations apply to the other components of the sample collection device.

FIGS. 9 and 10 show the precision for the weighed portion of the feces. The associated explanations can be found in Example 8 (supra).

FIGS. 11A and 11B show measurement results obtained with the sample preparation system according to the invention (FIG. 11 a) and measurement results obtained with the prior art sample preparation system (FIG. 11b).

1. A kit for in vitro diagnostics, comprising
a) at least one sample preparation device for feces samples, with a closeable vessel for receiving a liquid reagent or/and solvent and a cylindrical sample collection rod with a diameter d of 1-2 mm, which has in the region of one of its ends on its peripheral surface at least one scoop-type recess and is insertable with this end into the vessel through an insertion opening of the vessel provided with a stripping shoulder, characterized by that
the at least one scoop-type recess represents an annular or ring segment-shaped recess of the cylindrical sample collection rod, wherein the angle α between the lateral surface of the sample collection rod and the recess is approximately rectangular, and wherein the angle θ between the base surface of the recess and the core cylinder also is approximately rectangular, and wherein the total volume of the recess is 10-100 μl,
wherein the closeable vessel 1-2 ml comprises a buffer solution, wherein the buffer solution comprises a phosphate-buffered sodium chloride solution with a pH value of 6.9-8, Tween 20, sodium azide <0.05% and the detergent CHAPS (3-[(3-cholamidopropyl)dimethylammonio]-1-propanesulfonate, 10 mM and
b) an immunological device for diagnosis and exclusion of a pancreatic insufficiency in the feces,
wherein the device comprises a solid phase, to which is bound at least one antibody, selected from the group of the elastase 1-specific monoclonal antibodies capable of binding, which do not cross-react with animal pancreatic elastases.

2. The test kit according to claim 1, wherein the cylindrical sample collection rod comprises two to six annular or ring segment-shaped recesses as scoop-type recesses.

3. The test kit according to claim 1, wherein the cylindrical sample collection rod comprises four ring segment-shaped recesses as scoop-type recesses, wherein the ring segment spans 180° of the cylinder cross-section, and wherein the total volume of the four ring segment-shaped recesses is 50 μl.

4. The test kit according to claim 1, wherein the device is designed in the manner of an ELISA test, wherein the antibody is bound in a microporous flat membrane or hollow fiber membrane.

5. The test kit according to claim 1, wherein the device is designed in the manner of an immunoassay.

6. The test kit according to claim 3, wherein the device is designed in the manner of a lateral flow immunoassay.

7. The test kit according to claim 1, wherein all antibodies are monoclonal antibodies.

8. The test kit according to claim 1, wherein the antibody of clone 1 is used.

9. The immunological test kit according to claim 1, for diagnosis and exclusion of an exocrine pancreatic insufficiency in the feces or an acute pancreatitis in the serum.
